(19) United States
(12) Patent Application Publication
(10) Pub. No.: US 2023/0284893 A1
(43) Pub. Date: Sep. 14, 2023

(54) Gynecology and urology kit for improved bladder visualization for fast fill catheter

(71) Applicant: FarWest Land Management Group LLC, El Paso, TX (US)

(72) Inventor: Richard Farnam, El Paso, TX (US)

(73) Assignee: FarWest Land Management Group LLC, El Paso, TX (US)

(21) Appl. No.: 18/005,658

(22) PCT Filed: Jul. 16, 2021
(86) PCT No.: PCT/US2021/042073

§ 371 Date: Jan. 16, 2023

Related U.S. Application Data
(60) Provisional application No. 63/052,921, filed on Jul. 16, 2020.

(51) Int. Cl. A61B 1/307; A61M 39/22; A61B 1/015

(52) U.S. Cl. CPC A61B 1/307; A61M 39/223; A61B 1/015; A61M 2039/229; A61M 2205/6081; A61M 2210/1085

(57) ABSTRACT

The present invention is a kit designed with a novel sized catheter valve capable of directing fluid easily through two separate paths or restricting fluid passage completely. The novel catheter valve is easy for an operator to use due to the relatively large valve control. The novel valve catheter also has a color code as an added safety measure.

### CROSS-REFERENCE TO RELATED APPLICATIONS

This application claims priority and benefit of U.S. provisional patent application No. 63/052,921 filed on Jul. 16, 2020 and priority to PCT/US21/42073 with a filing date of Jul. 16, 2021.

### BACKGROUND

Pelvic surgery including hysterectomy and related urologic surgical procedures account for one million procedures per year in the United States alone.

### TECHNICAL FIELD

This invention relates to a medical device kit, particularly a kind of novel bladder distending kit to facilitate visualization of the bladder margin during pelvic surgery so as to prevent inadvertent bladder injury

### BACKGROUND TECHNOLOGY

There are many urology related procedures that require visualization of the bladder to facility accurate location and avoidance of injury to the human bladder and adjacent anatomy. In the process of some operative procedures that utilize open surgery, laparoscopy, or robotics (such as hysterectomy, Cesarean section and sacrocolpopexy) that require dissection of the bladder off of the cervix and vagina, the urinary bladder can be inadvertently injured. Cystotomy (bladder injury) is a rare recognized complication of hysterectomy, Cesarean section, and sacrocolpopexy and other pelvic surgeries. Current best practices require the operator to fill the bladder to visualize the anatomy and avoid injuring tissue unnecessarily. The present method for visualizing the bladder and adjacent anatomy includes a time consuming process of gathering separate components including cystoscopy tubing, Toomey syringe, and distention medium (usually sterile milk or normal saline). The Toomey syringe and or cystoscopy tubing must then be filled with the distention medium and then the medium is then injected into the bladder via an indwelling foley catheter. The bladder is then drained when distention is no longer required. When the bladder wall is distended the operator can more clearly delineated the margin between the bladder and vagina and cervix, thus informing a safer dissection and decreasing the risk of inadvertent cystotomy. The operating room nurse will be occupied with the process of assembling the components of this bladder retrofilliing assembly and filing and emptying the bladder. An operating room nurse is required to measure the infused and drained fluids to confirm that urine output is appropriate for patient health during the procedure. In absence of a standardized system of ins-tilling bladder fluid urinary output can be difficult to calculate accurately, Additionally, the presently available setup requires three separate sterile packages to be opened and assembled. The steps required to set up the present system requires a lot of the operating room nurse and operator time to setup, assemble sterile in the sterile field, and creates and additional amount of unnecessary medical waste.

It is also recognized and recommended in clinical practice after pelvic surgeries to conduct an assessment of the bladder and ureters to ensure that no damage to these organs has occurred. In many cases an optimal way for making this assessment is the use of a cystoscope where the internal surface of the bladder can be viewed as well as the vesicoureteral junction. The vesicoureteral junction can be observed to look for flow of urine into the bladder indicating that no impairment of the ureters, bladder, or other structures has occurred during the surgery. In some cases, use of an intravenous dye such as indigo carmine can be used to visualize urinary tract flow and determine possible leakage, However these finding may help identified bladder and ureter injuries after fact. Therefore cystoscopy may increase detection of injury, but does not prevent injury. The incidence of urologic injury has been reported to range from 1 to 3%, During laparoscopic hysterectomy, the incidence of ureteral injury has been reported at 0.2 to 4.3%, Bladder laceration, thermal injury and bladder tearing commonly occur due to poor visualization of the vesicovaginal space (the anatomic space between the bladder and anterior vagina). A convenient, easy to use, cost-effective bladder distention tool could reduce these types of bladder injuries. The current method of bladder distention is not standardize, not readily available, requires assembly of separate components, causes expensive operating room surgical delays, consumes valuable operating room staff time, and confounds accurate urinary output measurement. Whereas postoperative universal cystoscopy has definitive benefit for injury detection, it does not prevent injury to urinary tract. Furthermore absence of a standardized bladder filling apparatus may result in repeated cystoscopy and multiple instances of urethral instrumentation, increasing risk of infection and urethral trauma. Systems and methods as disclosed herein can obviate these shortcomings.

In certain bladder or prostate surgeries, continuous bladder irrigation is used. This is achieved by introducing fluid continuously into the bladder and simultaneously draining the bladder. This type of Foley catheter is called a three-way catheter. One port will serve as fluid irrigation port into the bladder. The second port is for continuous drainage of the bladder contents into a large urine bag. The third port is for a valve mechanism where a balloon is inflated inside the bladder to keep the tip of the Foley catheter indwelling inside the bladder.

In certain patients, the bladder must be drained for many years, such as debilitated patients or those with neurological or spinal cord lesions. If the bladder is not drained, the pressure in the bladder will build up and the kidneys will be obstructed. Continuous bilateral kidney obstruction may lead to renal failure.

The present disclosure relates to systems, devices, and methods to treat a bodily organ and disorders thereof. In particular, the present disclosure describes improved surgical kit setup and methods for treating a urinary bladder or other bodily organ by including sterile saline, multi-port stopcock, bladder irrigation kit or cystoscopy tubing, and Foley Catheter.

The present invention is used for intraoperative bladder filling to delineate the margins of the bladder to prevent inadvertent injury from surgical dissection. The invention essentially consists for four components; 1) standard 2 channel 16 fr catheter with drainage bag, 2) a novel three-way or standard four-way adapter with stopcock for 3 position (drain, fill, occlude), 3) standard cystoscopy tubing, and 4) standard 1000 cc sterile water bag.

To date, no such kit or “out of the box” product exists to instantaneously intraoperatively distend the bladder (to improve visualization of the bladder anatomy), clamp the flow (to maintain the distention during dissection), and drain (after dissection is completed).

New research supports that interstitial cystitis (IC) is much more prevalent than originally thought. The total and the proportion of men with IC is much higher than originally estimated. Women: 3 to 8 million women in the United States may have IC. That is about 3 to 6% of all women in the US. Men: 1 to 4 million men have IC as well, however, this number is likely lower than the true rate because IC in men may often be mistaken for another disorder, such as chronic prostatitis/chronic pelvic pain syndrome. Children: Many adults comment that their IC symptoms started in childhood. Pediatricians also diagnose children with this chronic condition. But, epidemiology studies have not yet been done to estimate the true prevalence of children with IC.

Source: J. Quentin Clemens. Male Pelvic Pain. International Pelvic Pain Society 18th Annual Scientific Meeting on Chronic Pelvic Pain

Statistics for women are based on the findings of the RAND IC Epidemiology Study (RICE), the largest IC epidemiology study ever undertaken. This survey of more than 100,000 US households told us that from 2.7 to 6.5 percent of US women may have IC, which translates to about 3 to 8 million women. On the landmark IC studies page, there is a summary of the findings and poster session presented at the 2009 American Urologic Association meeting.

Prevalence estimates for men are based on the findings of the Boston Area Community Health (BACH) survey conducted in 2002 of residents in the Boston metropolitan area. Researchers interviewed some 6,000 men and women, ages 30 to 79, in their homes. One third of the randomly sampled population was African American; one third, Hispanic; and one third, Caucasian. At the 2008 AUA annual meeting and in the Journal of Urology, investigators revealed that many more men appear to have IC than initially thought. There's a summary of the BACH findings on the landmark IC studies page.

Your doctor will not perform your cystoscopy with hydrodistention in his or her office. The bladder needs to be filled to a high pressure in order to see the typical abnormalities of IC, a pressure that can cause significant pain if you are not anesthetized. In addition, in-office cystoscopy may not reveal the glornerulations on your bladder and the diagnosis of IC can be missed. Cystoscopy with hydrodistention is an outpatient surgical procedure. You will be admitted to the hospital, but in most cases, you will be discharged the same day as the procedure. Prior to this procedure, you may have basic blood work done, just as you would before any surgery. During the procedure you will receive general anesthesia, or your pelvic region will be numbed by spinal nerve-blocking agents, You will not feel any pain during the procedure. Your physician will insert a cystoscope through your urethra and into your bladder. This “scope,” which is fitted with a miniature camera, enables your physician to see the interior surface of your bladder to look for Hunner's ulcers and other abnormalities. Your doctor will first examine your bladder by viewing it through the cystoscope. After the initial cystoscopic examination, your physician will “hydrodistend” your bladder by filling it with fluid at a low pressure (60 to 80 centimeters) and keeping it full for one to five minutes before letting the fluid out. This process causes the bladder wall to stretch out (distend), allowing your physician to re-inspect your bladder with the cystoscope.

If you have IC, your physician typically sees irritated areas (glomerulations) on the bladder wall. Also, the hydrodistention allows your doctor to check your bladder capacity under anesthesia. This can be an indicator of the need for more aggressive therapies. The cystoscope may be fitted with instruments for obtaining biopsies of your bladder wall. While biopsy is not necessary for the diagnosis of IC, it is useful in ruling out bladder cancer. A bladder biopsy can also be helpful in determining if there are increased numbers of mast cells in the bladder wall, which may support the diagnosis of IC and indicate treatment options. Your doctor should not perform any additional treatment measures, such as bladder instillations, during this procedure, since your bladder will be very vulnerable from the procedure.

### RELEVANT ART

U.S. Pat. No. 7,988,615 issued to Anderson discloses A Surgical instrument for treating incontinence includes a handle portion elongate along a handle axis and a needle portion connected to the handle portion. The needle portion has a spacer portion along the handle axis, and has a distal end. The needle portion includes a substantially helical portion that is a variable spiral portion, extending from the straight spacer portion. The variable spiral portion is sized and shaped to extend from an incision Substantially adjacent the patient's obturator foramen through the obturator foramen along a path in a region between the Superior pubic ramus and the inferior pubic ramus. The needle portion has a structure near the distal end that associates the instrument with an implantable material configured to treat incontinence. U.S. Pat. No. 9,149,176 issued to Greenberg et al. discloses systems and methods of cannulating a body orifice such as a bladder are disclosed. A cystoscopy catheter can include a proximal end, a distal end having at least one exit port, and a flexible elongate tubular body therebetween; an inflatable balloon near the distal end of the catheter, the balloon transformable from an unexpanded and an expanded configuration; a first proximal port for fluid inflow; a second proximal port for fluid outflow; a third proximal port for balloon inflation, in fluid connection with the balloon; and a fourth proximal port for insertion of a diagnostic and/or therapeutic instrument such as a cystoscope therethrough, wherein the first, second, and fourth proximal ports are fluidly connected to a common central lumen extending through the elongate tubular body to the exit port on the distal end.

U.S. Pat. No. 8,414,562 issued to House discloses an indwelling urinary catheter assembly is disclosed having an indwelling catheter and a sheath enclosing an insertable portion of the indwelling catheter.

U.S. Pat. No. 9,492,631 issued to Tanghoj discloses a catheter assembly includes a urinary catheter and a package provided to contain the catheter. The catheter has a mid-section connected between a proximal end and a distal end. The proximal end of the catheter is adapted for insertion into a urinary canal and the distal end of the catheter is connected to a seal. The package includes a proximal compartment section that is removable from a distal section of the package to provide an outlet in the distal section of the package. The catheter is adapted to move relative to the distal section of the package such that (a) the seal at the distal end of the catheter slides relative to the distal section of the package and (b) at least the mid-section of the catheter is movable through the outlet in the distal section of the package.

U.S. Pat. No. 9,795,761 issued to Lockwood et al. discloses printed instructions can be included with the tray in a medical procedure kit. The printed instructions can include instructional material, such as pictorial, step-by-step instructions intended for a health care services provider for using the medical procedure kit. The printed instructions can also include a separate patient aid, suitable for inclusion within the medical procedure kit, which includes patient education information relating to a particular medical procedure. To make recognition easier, the patient aid can be configured with a greeting card appearance, activity sheet appearance, or other graphical indicia that indicates that the patient aid is intended for the patient and should be delivered to the patient.

US20030092689A1 publication discloses a kit for treating a human male, including a means for necrosing prostate tissue, an antiandrogen drug, and a means for administering the antiandrogen drug. A kit including a first surgical device for delivering a chemoablation fluid to prostate tissue transurethrally, an antiandrogen drug such as bicalutamide, and a second surgical device for administering the antiandrogen drug.

US20060009742A1 publication discloses a The present invention relates to an ergonomic urological catheterization/irrigation tray. The tray includes a tray structure having a recessed area that includes at least a bottom, opposite side walls, a front and a back, wherein the front has a first width of a predetermined dimension and the back has a second width of a predetermined dimension less than the first width, such that the recessed area is wider at the front than it is at the back. The recessed area has at least one drain located at a lower area thereof. The tray further includes a top flange extending at least outwardly each of the opposite side walls, wherein a user may rest the flange on front upper portions of legs when in a supine position, for urinary bladder evacuation into the tray. Surgical kits contain the aforementioned tray and specified components.

US20140220094A1 publication discloses Glycerophosphate salts have been found to drastically improve a laparoscopic surgery by reducing the inflammatory response inflicted by the surgery and enhancing wound healing. Methods and devices for improving the laparoscopic surgery using a composition comprising an effective amount of a glycerophosphate salt are described.

These publications and all other referenced patents are incorporated herein by reference in their entirety. Furthermore, where a definition or use of a term in a reference, which is an incorporated reference here, is inconsistent or contrary to the definition of that term provided herein the definition of the term provided herein applies and the definition of that term in the reference does not apply.

### SUMMARY

The present invention addresses the clinical need for an out of the box product to instantaneously intraoperatively distend the bladder (to improve visualization of the bladder anatomy), damp the flow of fluid (to maintain the distention during dissection), and drain fluid (after dissection is completed).

Still further advantages will become apparent from a study of the following description and the accompanying drawings.

Useful in Laparoscopic or robotic hysterectomy, Burch (incontinence surgery), Sacrocolpopexy (prolapse surgery), C-section, Bladder resection, and Endometriosis surgery,

### BRIEF DESCRIPTION OF THE DRAWINGS

FIG. 1 Urinary bladder with F.A.S.T. three position catheter valve linking a standard catheter drainage bag and standard cysto tubing along with a distal dual lumen spline anchor

FIG. 2 cross section of dual lumen distal for fill and drain lines of different relative size.

FIG. 3 F.A.S.T. three position valve is a present invention for quickly changing fluid dynamics with a switch toggle.

FIG. 4 is a close up view of F.A.S.T. three valve showing the drain, clamp, and fill position with color coding to increase ease of use and safety.

FIG. 5A illustrates a preferred embodiment of the FAST with out flow alignment.

FIG. 5B illustrates a cross-section view of the preferred embodiment of the FAST with out flow alignment.

FIG. 6A illustrates a preferred embodiment of the FAST with in flow alignment.

FIG. 6B illustrates a cross-section view of the preferred embodiment of the FAST with in flow alignment.

FIG. 7A illustrates a preferred embodiment of the FAST with stop flow alignment.

FIG. 7B illustrates a cross-section view of the preferred embodiment of the FAST with stop flow alignment.

FIG. 8 illustrates a commercially available Foley Catheter.

FIG. 9 illustrates commercially available tubing for controlling fluid flow via a Foley Catheter.

FIG. 10 illustrates a commercially available bag for fluid collection via a Foley Catheter.

FIG. 11 illustrates a commercially available bag for two-way fluid collection and delivery via a Foley Catheter.

### DETAILED DESCRIPTION

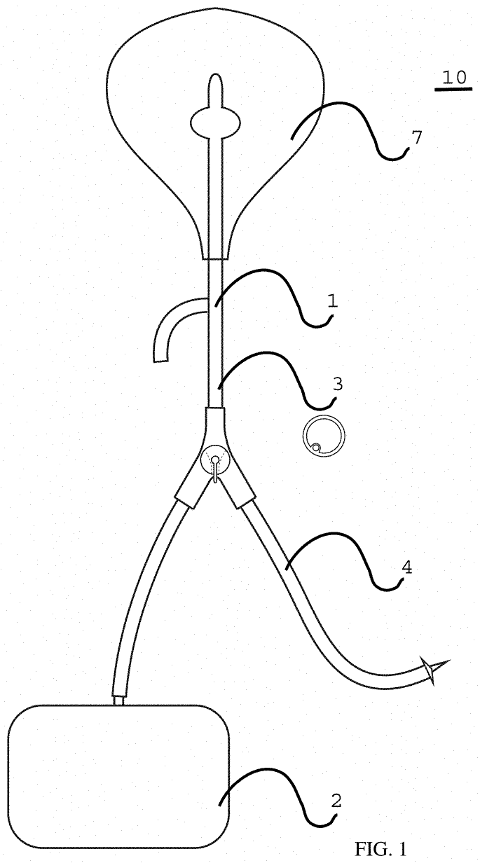

FIG. 1 is an overview of the kit with a Foley Catheter 1 with standard two channel 16fr lumen; a drainage bag 2; a three or four-way adapter 3 with stopcock for three position (drain, fill, occlude); and, standard cystoscopy tubing 4. This is the most basic setup. Foley catheters are very well known in the medical and surgical fields.

The inventive aspect of the present invention is two-fold. First the F.A.S.T. catheter valve 3 and second the arrangement of the other commercially available device portions with the F.A.S.T. catheter valve 3 in a single kit 10. The kit 10 can be sterile and arranged for quick assembly in the operating room in or out of the sterile field. The F.A.S.T. catheter valve 3 has a urine outflow portion 8 that is adapted for standard Foley drainage bag 2. The urine outflow portion 8 can be characterized as having a female shaped adapter for secure fluid sealed attachment to a urine collection bag or drainage bag 2. The F.A.S.T. catheter valve 3 also has a cysto attachment portion 9 that is adapted for standard cysto tubing 4 for irrigation of the bladder. The cysto attachment portion 9 can be characterized as having a female shaped adapter for secure fluid sealed attachment to cysto tubing for irrigating a bladder (not shown).

FIG. 1 shows the F.A.S.T. catheter valve 3 also has a Foley attachment portion 11 that is adapted for attachment to a standard Foley catheter 1. The Foley attachment portion 11 can be characterized as having a male shaped adapter for secure fluid sealed attachment to the Foley catheter tip 7 to be disposed in the bladder.

The F.A.S.T. catheter 3 valve also has a valve control 12 that can be placed in one of three positions, fill, stop, or drain by an operator applying pressure to the valve control 12 typically by pinching between a finger and thumb and rotating the valve control 12 relative to the urine outflow portion 8 and cysto attachment portion 9. When the valve control 12 is aligned over the urine outflow portion 8, urine will be drained from the bladder to the drainage bag 2.

When the valve control 12 is aligned over the cysto attachment portion 9, fluid will flow to the bladder via the cysto tubing 4, through the Foley catheter 1 and finally exit into the bladder via Foley catheter tip 7.

When the valve control 12 is aligned with the Foley attachment portion 11, no fluid will pass through the F.A.S.T. catheter 3.

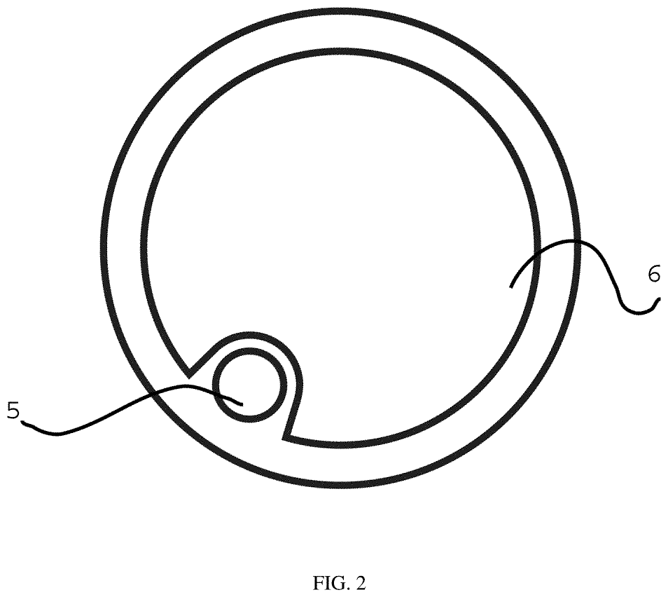

FIG. 2 Shows the cross-section of a two way urinary catheter lumen for infusion lumen 5 for delibery of fluid through Foley catheter tip 7 and eventual fluid removal through drain lumen 6.

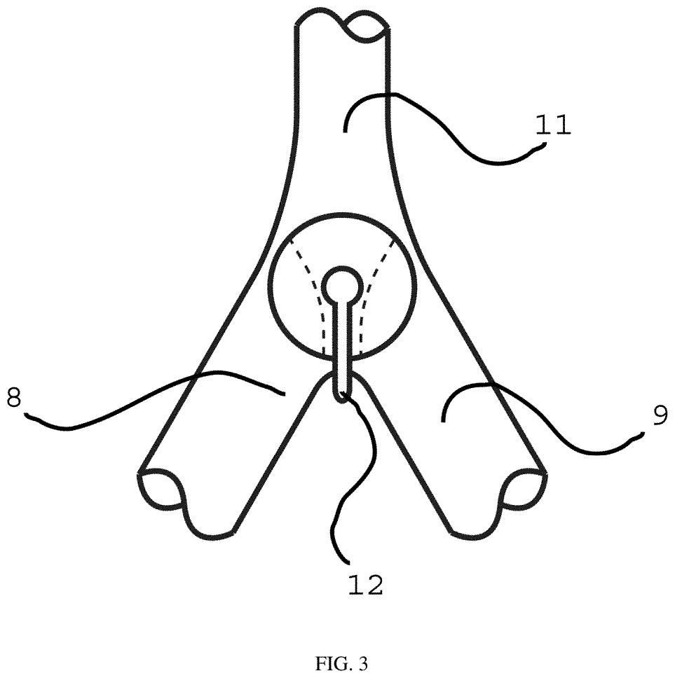

FIG. 3 shows an independent and unique aspect of the present invention, a F.A.S.T. three position catheter valve comprising a urine outflow portion 8, a cysto attachment portion 9, a Foley attachment portion 11, and a valve control 12.

FIG. 4 shows a close up view of the F.A.S.T. three position catheter valve 3. In a preferred embodiment the urine outflow portion 8 would be yellow to indicate that urine was the intended outflow. The cysto attachment portion 9 would be colored blue to indicate that water or non-urine fluid would flow. Additionally, in the preferred embodiment a portion between urine outflow portion 8 and the cysto attachment portion 9 would be colored red such that when the control valve 12 was aligned with the red indication portion it would be indicate to the oipreator that no fluid should flow through the catheter valve 3.

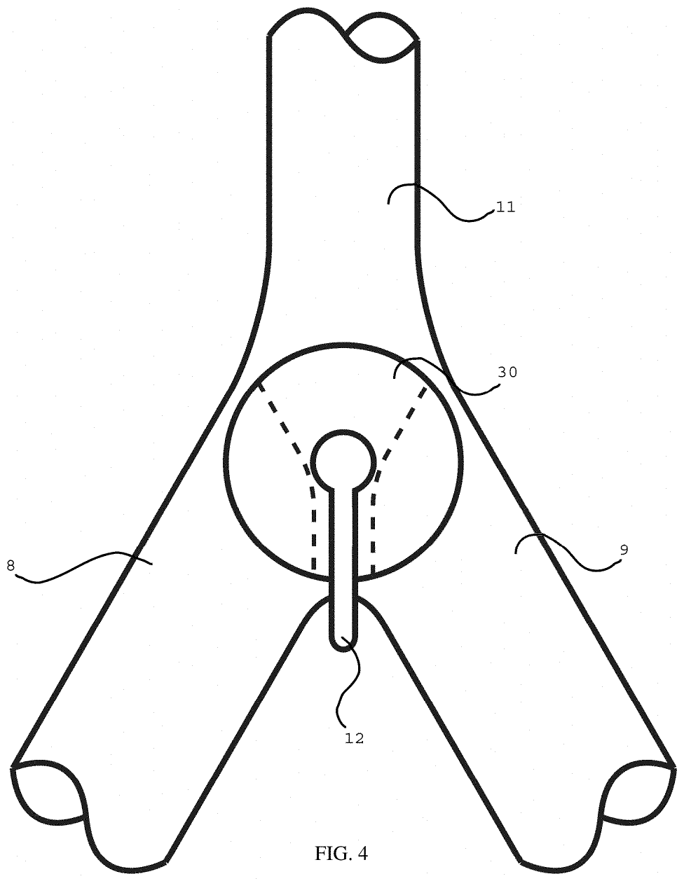

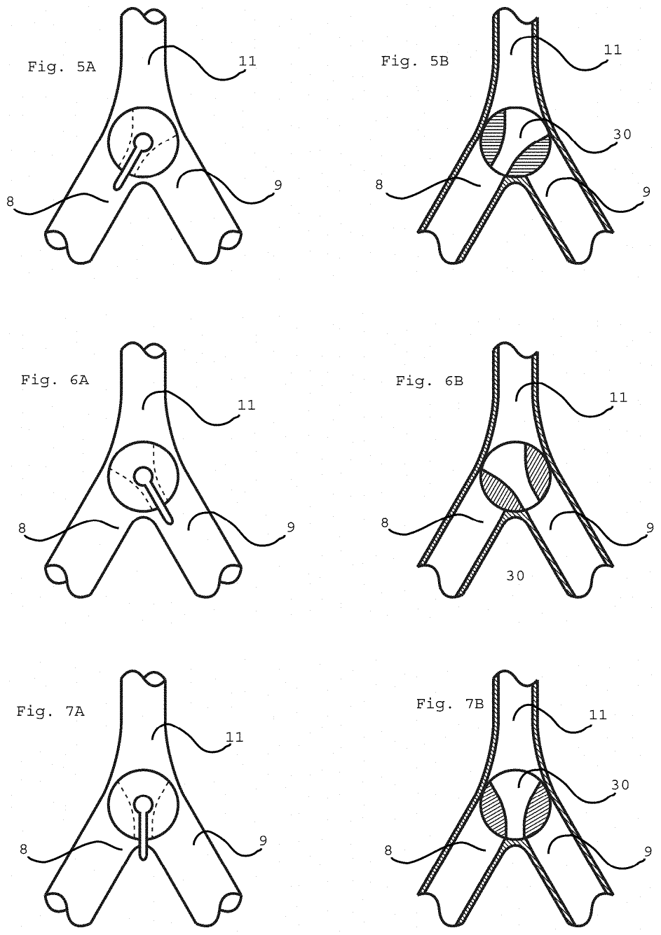

FIGS. 5A-5B shows the F.A.S.T. three position catheter valve in the drain position. Specifically, FIGS. 5A-5B show the F.A.S.T. three position catheter valve 3 with the valve control 12 in alignment with urine outflow portion 8 while the Foley attachment portion 11 creates a through path 30 from the bladder. FIG. 5B shows a cutaway view through the shows the F.A.S.T. catheter valve 3. When the valve control 12 is in fluid communication between urine outflow portion 8 and Foley attachment portion 11, the cysto attachment portion 9 is sealed off and no fluid flows in or out through cysto attachment portion 9.

FIGS. 6A-6B shows the F.A.S.T. three position catheter valve in the fill position. Specifically, FIGS. 6A-6B show the F.A.S.T. three position catheter valve 3 with the valve control 12 in alignment with cysto attachment portion 9 while the Foley attachment portion 11 creates a through path 30 to the bladder. FIG. 6B shows a cutaway view through the shows the F.A.S.T. catheter valve 3, When the valve control 12 is in fluid communication between cysto attachment portion 9 and Foley attachment portion 11, the urine outflow portion 8 is sealed off and no fluid flows in or out through cysto attachment portion 9. The fluid only can only be directed into a patient bladder out the Foley catheter tip 7.

FIGS. 7A-73 shows the F.A.S.T. three position catheter valve in the clamped or stop position. Specifically, FIGS. 7A-7B show the F.A.S.T. three position catheter valve 3 with the valve control 12 in alignment with neither urine outflow portion 8 nor cysto attachment portion 9 so that there is no through path 30 between the patient bladder via Foley attachment portion 11 to move fluid in or out of the patient with the catheter valve 3. FIG. 7B shows a cutaway view through the shows the F.A.S.T. catheter valve 3. Typically, a patient would have their bladder filled and viewed by the surgeons, nurses, and associated clinicians. The bladder would be filled or drained during surgery to facilitate the visualization of the bladder relative to other tissue.

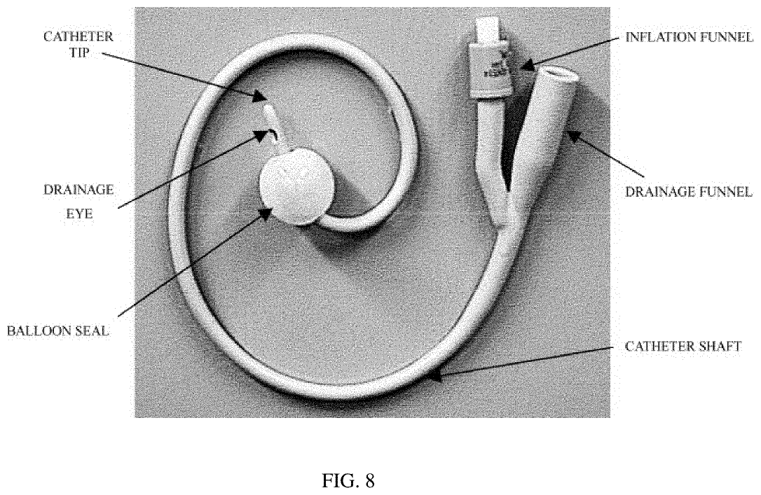

FIG. 8 Commercially available Foley Catheter.

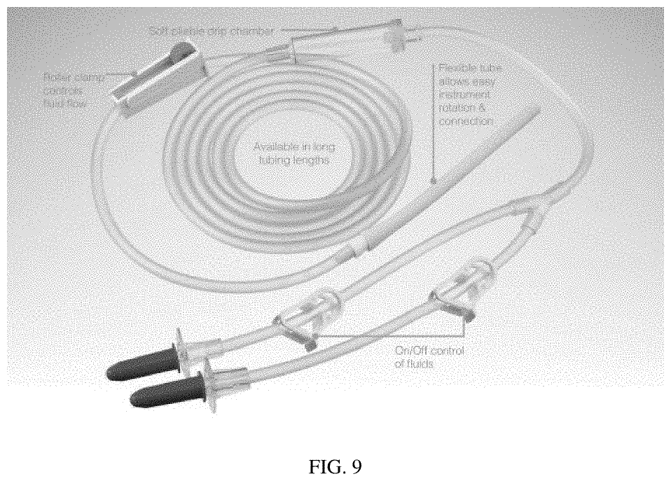

FIG. 9 Commercially available tubing for contolling fluid flow from a Foley Catheter.

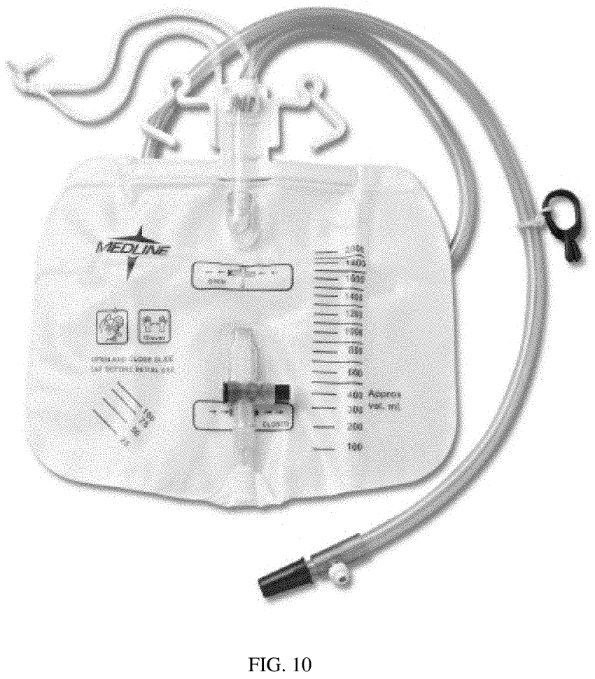

FIG. 10 Urinary Drainage Bag with Anti-Reflux Valve 2,000 mL—DYNC1674

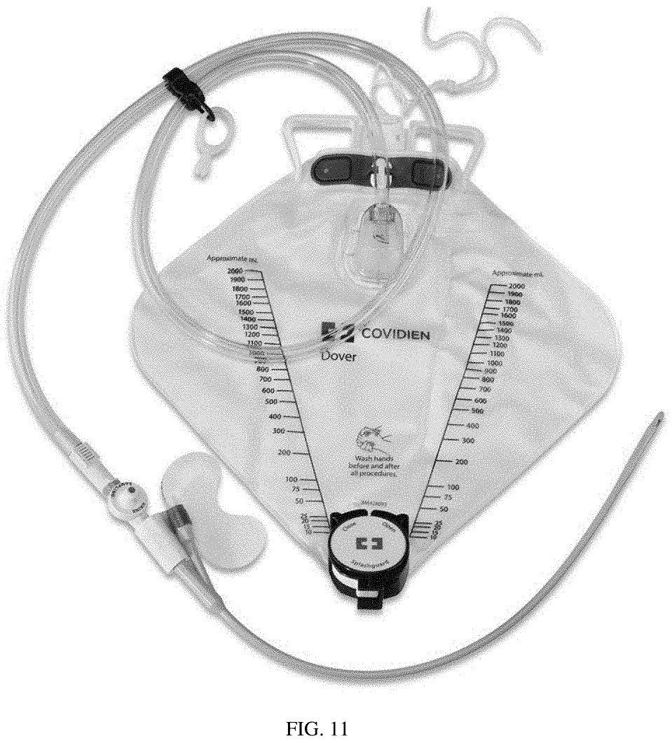

FIG. 11 Commercially available Covidien Dover 2-Way Foley Catheter Tray With Drain Bag Insertion/Irrigation Trays.

Foley constant drain, toggle for fill separate from the bag would be hanging on an IV pole, still give it a squeeze, regular height, diagnostics at 60-80 cm/H2O is the pressure in cycto tubing 4 when delivering fluid, Fluid can be delivered more quickly by increasing pressure applied to the fluid bag. Various manufacturers have warnings for appropriate delivery pressure,

FIG. 8 illustrates a commercially available Foley Catheter.

FIG. 9 illustrates commercially available tubing for controlling fluid flow via a Foley Catheter.

FIG. 10 illustrates a commercially available bag for fluid collection via a Foley Catheter.

FIG. 11 illustrates a commercially available bag for two-way fluid collection and delivery via a Foley Catheter.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What is claimed is:

1. A medical kit for visualization of human anatomy comprising: a standard 16 French diameter Foley catheter connected to a drainage bag with the connection at a proximal portion of the Foley catheter and wherein a three-way adapter stopcock is connected at a proximal portion of the Foley catheter and a proximal portion of a standard cystoscopy tubing wherein the cystoscopy tubing is sterile; and the kit does not include the distention medium, as a standard 1000 cc sterile water or osmotically balanced fluid for human use in a bag are readily available.

2. The medical kit of claim 1 wherein the three-way adapter stopcock is adapted with male to female connector on a distal portion intended to connect to the proximal portion of the Foley catheter and the distal portion of the three-way adapter stopcock has two female to male adapters on a proximal portion and a control lever centrally disposed on the three-way adapter stopcock to mechanically control fluid flow direction.

3. The medical kit of claim 1 wherein the three-way adapter stopcock is further color coded with the male to female connector on the distal portion uncolored or clear and a fill position designated blue or green in color to designate flow in the distal direction to a patient, a clamp/closed position designated red in color to indicate no fluid flow to or from a patient, and a yellow designated portion to indicate proximal fluid flow away from a patient.
